(19) United States
(12) Patent Application Publication
(10) Pub. No.: US 2008/0275508 A1
(43) Pub. Date: Nov. 6, 2008

(54) Bone end (epiphysis) fracture fixation device and method of use

(76) Inventor: George J. Haidukewych, Tampa, FL (US)

Correspondence Address: DAVID W. PETTIS, JR., PA, 1210 MILLENIUM PARKWAY, SUITE 2010, BRANDON, FL, 33511-0833, US

(21) Appl. No.: 11/897,647

(22) Filed: Aug. 31, 2007

Related U.S. Application Data
(60) Provisional application No. 60/927,667, filed on May 4, 2007.

(51) Int. Cl. A61B 17/70; A61B 17/56; A61B 17/58

(52) U.S. Cl. 606/280; 606/65; 606/286

(57) ABSTRACT

A bone end (epiphysis) fracture device including a rotatable fixation plate having a guide pin aperture and a plurality of fixation screw apertures formed therethrough. The guide pin aperture is used by a surgeon to position the fixation plate so that one or more fixation screw apertures are properly positioned to receive fixation screws inserted by the surgeon for repairing a bone end fracture. In another embodiment, the fracture fixation device also includes a side plate to which the fixation plate is rotatably attached. This embodiment further includes one or more side plate apertures formed through an elongate extension, whereby the fixation device may also be attached to the bone for which an end fracture is being repaired. A lock screw is provided in this embodiment for locking the position of the fixation plate with respect to the side plate.

### PRIORITY CLAIMING

Applicant claims the benefit of his prior Provisional Application, Ser. No. 60/927,667, filed May 4, 2007, and titled FEMORAL NECK FRACTURE FIXATION DEVICE AND METHOD OF USE.

### BACKGROUND OF THE INVENTION

1. Field of the Invention

The present invention relates to an improved fixation device for fractures of the ends of bones (epiphysis), and is described in a preferred embodiment with respect to a femoral neck fracture, and its method of use is also disclosed.

2. Description of the Prior Art

The use of various plates and bone screws for fixing bone fractures is old and well known in the art. In fact, prior art patents teach numerous devices for fixing a fracture of bone ends such as the femoral neck. For example, U.S. Patent Application Publication No. US 2006/0217711 to Stevens. et al., discloses a locking plate that extends along the length of the femur and provides apertures for inserting a plurality of bone anchor screws into the femoral head and across the fracture. Similar structure is also disclosed in U.S. Patent Application Publication No. US 2005/0101959 to Mitkovic. U.S. Pat. No. 5,871,485 to Rao, et al., discloses and teaches yet another device for fixing a fracture of the femoral head. Yet another device, also employing a fixation screw that extends into the femoral head across the fracture is disclosed in U.S. Pat. No. 6,139,552 to Horiuchi.

It is therefore quite clear that prior art patent literature teaches a variety of devices and attendant methods for surgically fixing fractures of the femoral neck. Just as apparent is the fact that these prior art devices possess virtually no means for adjusting a particular device to the needs of a particular patient. Variables such as size of the femur and the femoral head and the position and orientation of the femoral head fracture must be considered in the doctor's decision-making process with regard to placement and use of a fixation device. In addition to these variables, the doctor must also consider the health condition of the bone itself.

It is therefore clear that there remains a need in the art of femoral neck fracture fixation devices and methods of use for a device that is capable of adjustment to suit the needs of a particular patient. Such an adjustable device would not only prove to be more economical, but would also provide better care when fixation of a femoral neck fracture was needed.

It is also to be understood that this invention is not limited to fixation of femoral neck fractures, but is adaptable to use for fixing a variety of bone end (epiphysis) fractures. The drawing figures and detailed description relate to a femoral neck fracture for the purpose of clearly describing preferred embodiments of the invention.

### SUMMARY OF THE INVENTION

The present invention relates to a bone end (epiphysis) fracture fixation device and its method of use. The fixation device of this invention is particularly characterized by its construction to permit radial adjustability of a fixation plate through which a plurality of fixation screws may be inserted for the purpose of fixing the bone end fracture. Means are also provided for aligning the device according to the patient's needs. In a second embodiment, the rotatable fixation plate is movably mounted on a side plate, whereby the device may also be attached to the patient's bone away from the end fracture, should that be deemed necessary by the doctor. A method for using the bone end (epiphysis) fracture fixation device of the present invention, in both a primary and secondary embodiment, is also disclosed with regard to a femoral neck fracture.

The invention accordingly comprises the features of construction, combination of elements, and arrangement of parts which will be exemplified in the constructions hereinafter set forth, as well as the several steps and the relation of one or more of such steps with respect to each of the others thereof, which will be exemplified in the methods hereinafter disclosed, for using the invention.

### BRIEF DESCRIPTION OF THE DRAWINGS

For a fuller understanding of the nature and objects of the invention, reference should be had to the following detailed description taken in connection with the accompanying drawings in which:

FIG. 1 is a plan view of a first preferred embodiment for the bone end (epiphysis) fracture fixation device of this invention.

FIG. 2 is a side elevation illustrating the placement of one fixation screw through the fixation plate.

FIG. 3 is a fragmentary view of a patient's femur having a neck fracture, illustrating the use of a guide pin to position the fixation plate.

FIG. 4 is a view similar to that of FIG. 3 illustrating the use of three guide pins for orientating the fixation plate of the present invention.

FIG. 5 is a view similar to that of FIG. 3 illustrating the use of three fixation screws in combination with the fixation plate to fix a femoral neck fracture.

FIG. 6 is a plan view of a second preferred embodiment for the device of this invention, including a fixation plate rotatably attached to a side plate.

FIG. 7 is a view similar to that of FIG. 5 showing use of the second preferred embodiment of this invention with three fixation screws operatively mounted across a femoral neck fracture and two side plate screws inserted into the patient's femur.

Similar reference characters refer to similar parts throughout the several views of the drawings.

### DETAILED DESCRIPTION

Referring to the view of FIG. 1, a first preferred embodiment of the bone end (epiphysis) fracture fixation device is generally indicated as 10. Device 10 comprises a substantially planar and substantially circular fixation plate 12. Use of the phrase “substantially circular” is meant to describe that the perimeter of fixation plate 12 is not to be limited to that geometry. Virtually any geometric shape could be used. In similar fashion, use of the phrase “substantially planar” is intended to indicate that fixation plate 12 might define a curved plane, depending upon the topography of the patient's bone to which the plate 12 is being applied.

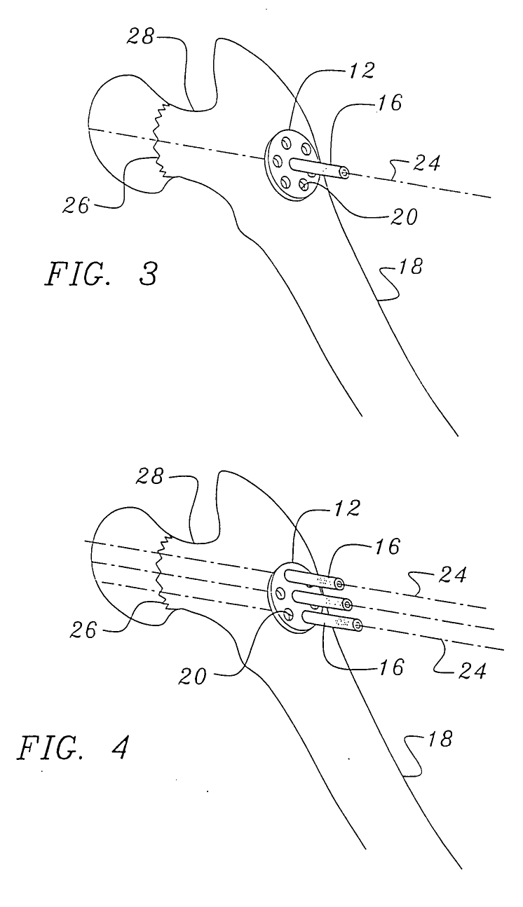

Still referring to the first preferred embodiment of FIG. 1, a guide pin aperture 14 extends through plate 12 and is used by the surgeon in combination with a guide pin 16 (FIG. 3) to position fixation plate 12 properly on the patient's femur 18 (FIG. 3).

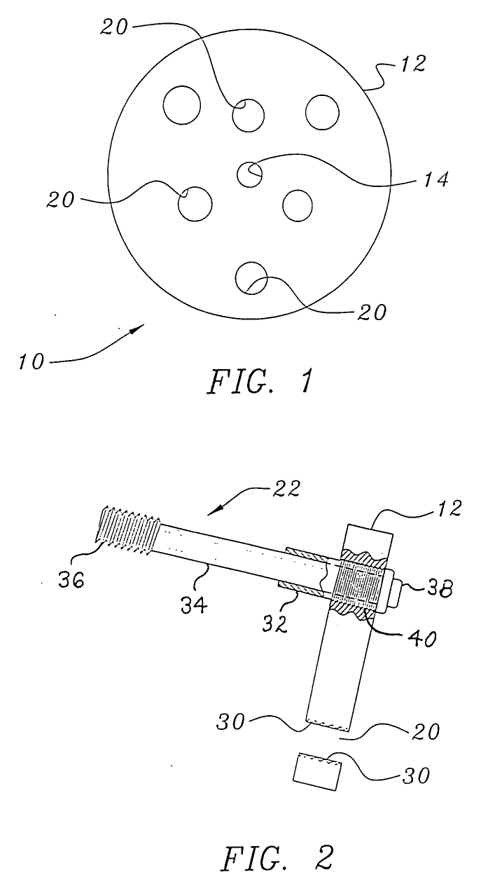

As also clearly seen in the view of FIG. 1, fixation plate 12 further comprises a plurality of fixation screw apertures 20, and each of these screw apertures 20 extend through fixation plate 12 whereby a fixation screw, generally indicated as 22 in the view of FIG. 2, may be inserted, as is more fully described below. While a plurality of fixation screw apertures 20 are provided through fixation plate 12, the exact size and relative placement of the illustrated screw apertures 20 are not to be interpreted as limiting factors to the scope of the present invention. The size of each of the screw apertures 20 is such that a fixation screw 22 may be operatively inserted therethrough. The relative placement of screw apertures 20 may define patterns other than the substantially triangular pattern of six screw apertures 20 as shown in FIG. 1.

Referring to the view of FIG. 3, one can see that a guide pin 16 has been attached to guide pin aperture 14, and a guide wire 24 has been inserted by the surgeon for initial placement of fixation plate 12 on femur 18. This procedure takes place following standard pre-op diagnosis to determine the location and nature of fracture 26 of the femoral neck 28 to provide proper initial placement of fixation plate 12. Once positioned as shown in FIG. 3, fixation plate 12 may be rotated about the axis defined by guide pin 16 and guide wire 24 in order to position one or more of the fixation screw apertures 20 for desired operative attachment of one or more fixation screws 22 (not shown in FIG. 3). Thus, one of the unique features of this invention is the ability of the surgeon to use a single fixation plate 12 to locate quite precisely the optimum positioning of one or more fixation screws 22. As shown in the view of FIG. 4, it is clearly within the scope of the present invention to employ a plurality of guide pins 16 and guide wires 24 in combination with not only the guide pin aperture 14 but also fixation screws apertures 20 to “lock” fixation plate 12 in position on the patient's femur 18 prior to insertion of fixation screws 22 as deemed appropriate by the surgeon.

As shown in the view of FIG. 5, after inserting the fixation screws 22, guide pins 16 and guide wires 24 are removed by the surgeon, and fracture 26 is “fixed.”

Considering the view of FIG. 2, one can see that the interior wall defined by each of the fixation screw apertures 20 is preferably threaded, as indicated at 30. Fixation screw 22 comprises a screw body 34 having a barrel 32 mounted thereon such that screw body 34 may rotate therein and such that the longitudinal axis of screw body 34 corresponds to the longitudinal axis of barrel 32. Distal end 36 of fixation screw 22 is threaded and proximal end 38 is appropriately configured to receive a tool (not shown) for the purpose of inserting and tightening screw body 34 to fix fracture 26 as best seen in the view of FIG. 5. Proximal end 40 of barrel 32 is threaded to be received and engaged by the threads 30 of fixation screw aperture 20. Thus, when barrel 32 is attached to a corresponding fixation screw aperture 20, and a corresponding screw body 22 is inserted through barrel 32, the alignment of screw body 34 is maintained as previously determined by the surgeon for proper placement across fracture 26 to secure and fix the femoral neck 28. While the view of FIG. 5 illustrates the use of three fixation screws 22, it is to be understood that one or more fixation screws 22 may be used within the scope of the present invention, depending upon the surgeon's evaluation of necessary steps to fix fracture 26.

It is, however, to be understood that the treads 30 of fixation screw apertures 20 are preferable, but not required. Of course, if the device were constructed to eliminate threads 30, there would be no need for threading proximal end 40 of barrel 32.

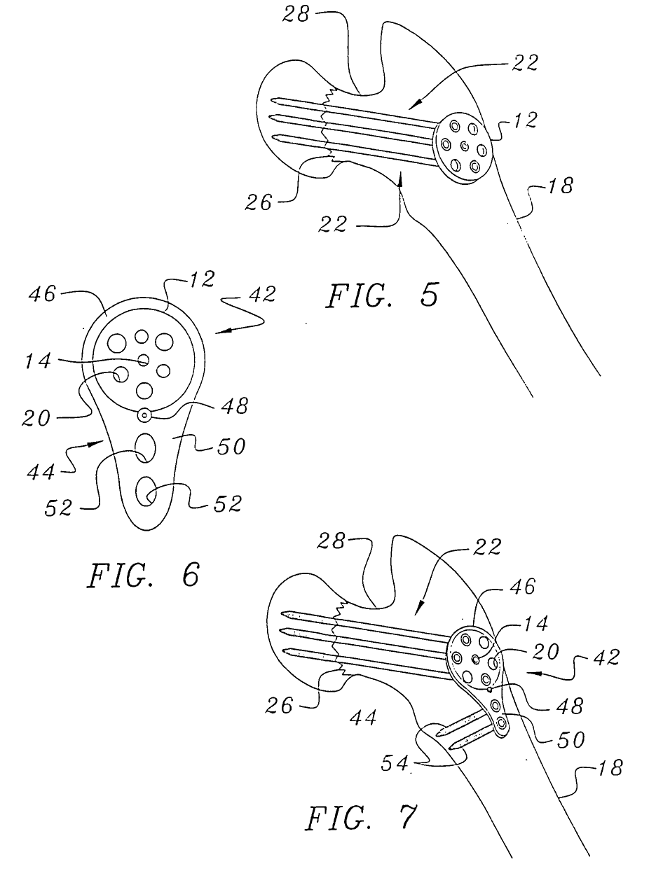

Attention is now invited to the views of FIGS. 6 and 7. These figures show a second preferred embodiment for the femoral neck fracture fixation device of this invention, generally indicated as 42. However, structural elements that are substantially identical to those described previously with regard to first preferred embodiment 10 are indicated by like reference numerals.

As clearly seen in the views of FIGS. 6 and 7. second preferred embodiment 42 comprises not only a fixation plate 12, but also a side plate generally indicated as 44. Fixation plate 12 of second preferred embodiment 42 is rotatably attached to enlarged end 46 of side plate 44. The rotational attachment of fixation plate 12 to enlarged end 46 is accomplished, preferably. by a rotatable attachment around guide pin aperture 14. It is also to be understood that enlarged end 46 of side plate 44 is relieved so that guide pin aperture 14 and the plurality of fixation screw apertures 20 extend through fixation plate 12 and through enlarged end 46. Because fixation plate 12 of the second preferred embodiment 42 is rotatably attached to side plate 44, second preferred embodiment 42 further comprises a lock screw 48 mounted on side plate 44 such that at least a portion of lock screw 48 will engage a portion of the perimeter of fixation plate 12 whereby tightening lock screw 48 will fix the relative position of the fixation screw apertures 20 with respect to the patient's femur. This permits the surgeon to position the fixation screw apertures properly, as determined in pre-op procedures, for insertion of the required number of fixation screws generally indicated as 22.

In both FIG. 6 and FIG. 7, one can also see that the elongated body 50 of side plate 44 comprises a plurality of side plate apertures 52 formed therethrough. With particular regard to the view of FIG. 7, it can be seen that side plate apertures 52 may be used to receive a corresponding side plate screw 54 therethrough and into the patient's femur, when necessary as determined by the surgeon, for the purpose of providing greater stability and strength to the second preferred embodiment 42 used for femoral neck fracture fixation.

Of course, it is to be understood that second preferred embodiment 42 is not to be limited to the particular geometric configuration shown in the views of FIGS. 6 & 7. In similar fashion, the scope of the present invention is not to be limited by the particular use of three fixation screws 62 or two side plate screws 54. Such specifics are determined by the surgeon after considering the patient's needs.

Finally, with regard to second preferred embodiment 42, it is also to be understood that an alternative rotatable attachment of fixation plate 12 to enlarged end 46 of side plate 44 may be accomplished wherein guide pin aperture 14, while extending through fixation plate 12. does not extend through enlarged end 46. In this construction, guide pin aperture 14 is used solely for the rotatable attachment of fixation plate 12 to enlarged end 46. Of course, with this type of construction, proper positioning of fixation plate 12 would be accomplished by the use of one or more guide pins 16 and corresponding guide wires 24 inserted through a corresponding one or more of the fixation screw apertures 20, substantially as shown in the view of FIG. 4.

Having thus set forth preferred embodiments for the femoral neck fracture fixation device of this invention, their method of use by the surgeon is substantially as follows. After normal pre-op investigation and determination by the surgeon, either first preferred embodiment 10 or second preferred embodiment 42 is selected and disposed on the patient's femur as illustrated in the several views of the drawings. Fixation plate 12 is positioned for the desired operative placement of fixation screws 22 and maintained in that position by the surgeon's use of one or more guide pins 16 and corresponding guide wires 24. With particular regard to second preferred embodiment 42, lock screw 48 would then be tightened.

Using standard surgical procedures, predetermined ones of the fixation screws 22 and, where appropriate, side plate screws 54, are attached by the surgeon and the incision is closed.

While the above detailed description has been made with regard to the use of preferred embodiments for fixing a femoral neck fracture. it is to be remembered that the scope of the present invention is not limited to fractures of the femoral neck. The fracture fixation device of this invention is suitable for use in a variety of conditions wherein a bone end (epiphysis) fracture is involved such as, for example only, a fracture involving the proximal humerus or a fracture of the distal femur.

It will thus be seen that the objects set forth above, among those made apparent from the preceding description, are efficiently attained and, since certain changes may be made in the constructions set forth and in carrying out the above method without departing from the scope of the invention, it is intended that all matter contained in the above description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Now that the invention has been described,

What is claimed is:

1. A bone end fracture fixation device used in combination with one or more fixation screws to repair fractures, said fixation device comprising: a fixation plate having a top surface and a bottom surface, said fixation plate being substantially planar and defining a substantially circular perimeter; said fixation plate further comprising a guide pin aperture extending therethrough from said top surface to said bottom surface and a plurality of fixation screw apertures extending therethrough from said top surface to said bottom surface in spaced apart relation to each other and to said guide pin aperture; an said fixation device further comprising a hollow guide pin comprising a cylinder having an outside diameter dimensioned and configured to be removably held within said guide pin aperture, whereby said fixation plate may be rotated about a central axis of said cylinder when said guide pin is inserted into said guide pin aperture.

2. A bone end fracture fixation device as in claim 1 wherein each one of said plurality of fixation screw apertures is threaded.

3. A bone end fracture fixation device as in claim 2 further comprising at least one fixation screw defined by a longitudinal body, said body comprising bone-engaging threads formed on a distal end and an enlarged head formed on a proximal end, said longitudinal body defining a body outside diameter; said fixation screw further comprising a hollow, cylindrical barrel having an inside barrel diameter dimensioned and configured to receive said screw body therein with said enlarged head at a proximal end of said cylindrical barrel; said proximal end of said cylindrical barrel being threaded to engage said threads of a corresponding one of said fixation screw apertures.

4. A bone end fracture fixation device as in claim 1 further comprising a side plate having a top surface and a bottom surface, said side plate being substantially planar and further comprising an enlarged end and an opposed elongate end, said fixation plate being rotatably attached to said enlarged end of said side plate such that said bottom surface of said fixation plate is adjacent said top surface of said enlarged end.

5. A bone end fracture fixation device as in claim 5 wherein said fixation plate is attached to said enlarged end at said guide pin aperture, whereby said fixation plate is rotatable around said guide pin aperture.

6. A bone end fracture fixation device as in claim 5 wherein said enlarged end is relieved from said side plate top surface through said side plate bottom surface so that a fixation screw may extend therethrough when the fixation screw is inserted through any one of said plurality of said screw apertures.

7. A bone end fracture fixation device as in claim 6 further comprising at least one side plate aperture formed in said elongate end and extending from said side plate top surface through said side plate bottom surface.

8. A bone end fracture fixation device as in claim 6 further comprising a lock screw operatively attached to said enlarged end, said lock screw comprising an enlarged head at its proximal end, said enlarged head being disposed in engaging relation to a portion of said top surface of said fixation plate when said lock screw is tightened, thereby preventing rotation of said fixation plate with respect to said side plate.
